(19) United States
(12) Patent Application Publication
(10) Pub. No.: US 2023/0380818 A1
(43) Pub. Date: Nov. 30, 2023

(54) SYSTEMS AND METHODS FOR CUTTING, REMOVAL, DISPLACEMENT AND RETENTION OF TISSUE SAMPLES

(71) Applicant: Miles Richie, Kennesaw, GA (US)

(72) Inventor: Miles Richie, Kennesaw, GA (US)

(21) Appl. No.: 18/202,201

(22) Filed: May 25, 2023

Related U.S. Application Data
(60) Provisional application No. 63/365,333, filed on May 26, 2022.

(51) Int. Cl. A61B 10/02

(52) U.S. Cl. CPC A61B 10/0233; A61B 2010/0208; A61B 2010/0225; A61B 10/0266

(57) ABSTRACT

A device that can perform excisions of tissue samples. The device may comprise an instrument body, a cutting and capturing assembly, and a biasing mechanism. The cutting and capturing assembly and the biasing mechanism can be configured to be releasably attached from the instrument body. The cutting and capturing assembly may be configured to have two fixtures, which can include a tubular sharp-edged tool and a capturing mechanism. At least a portion of the cutting and capturing assembly may be housed in the instrument body. In vivo, the device can create an incision in the tissue sample, which may be performed by the tubular sharp-edged tool. The capturing mechanism can be extended toward the incision, which can occur upon receiving an extension signal from the biasing mechanism. The capturing mechanism can capture the tissue sample from the incision, retract into the instrument body, and eject the retained tissue sample.

### CROSS-REFERENCE TO RELATED APPLICATIONS

This application claims the benefit of U.S. Provisional Application Ser. No. 63/365,333, filed on 26 May 2022, which is incorporated herein by reference in its entirety as if fully set forth below.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### SEQUENCE LISTING

Not Applicable

### STATEMENT REGARDING PRIOR DISCLOSURES BY THE INVENTOR OR A JOINT INVENTOR

Not Applicable

### BACKGROUND OF THE DISCLOSURE

### 1. Field of the Disclosure

The present invention relates generally to biological tissue sampling, and more particularly to a device capable of incising, capturing, displacing, and retaining tissue samples providing practitioners an ascendant manner to perform tissue sampling and methods therefore.

### 2. Background

There are a number of conventional ways for excising tissue samples. One of the primary reasons practitioners may obtain biological tissue samples is to perform a medical procedure known as a biopsy. A practitioner may perform a biopsy to analyze properties of the biological tissue sample. Analysis of excised tissue samples can provide diagnostic information about the tissue sample. Furthermore, the analysis can inform practitioners and medical teams in determining appropriate course of treatment for a patient. Conventional systems and methods for excising tissue samples can create an incision in the tissue sample but have no means of extracting the tissue sample after performing the incision using a singular device. For example, in the cases of excisional biopsies, a practitioner may create an incision on a patient's skin using one tool, such as a scalpel, and require another tool to extract the tissue sample, such as forceps.

Conventional systems and methods also lack the capability to consistently create uniform sized incisions in the tissue sample, retain the excised tissue sample, or eject the excised tissue sample after it has been cut using a singular device. Additionally, conventional systems and methods fail to meet the needs of the industry due to their inability to interchange fixtures on demand to perform tissue excision procedures. For example, a practitioner may need to change the size of the blade while performing an excisional biopsy due to varying textures and thickness within the tissue sample.

Thus, a need yet exists for systems and methods, capable of creating controlled, uniform incisions and extractions of tissue samples on demand by practitioners. The need is addressed by the present invention, and it is to such systems and methods that the present invention is primarily directed.

### BRIEF SUMMARY OF THE DISCLOSURE

In an exemplary embodiment of the present invention, a device may comprise an instrument body, a cutting and capturing assembly, and a biasing mechanism. The instrument body may comprise an activation end and an implementation end. The cutting and capturing assembly may be located proximally to the activation end of the instrument body. The cutting and capturing assembly may be configured to cut an element, capture the element, and displace the element, wherein at least a portion of the cutting and capturing assembly can be housed within the instrument body. The biasing mechanism may be configured to at least extend the cutting and capturing assembly from the implementation end of the instrument body upon reception of an extension activation signal.

In any of the embodiments disclosed herein, the cutting and capturing assembly and the biasing mechanism can be configured to be releasably attached to the instrument body.

In any of the embodiments disclosed herein, the implementation end of the instrument body can be configured to releasably attach fixtures.

In any of the embodiments disclosed herein, the cutting and capturing assembly can comprise at least two fixtures, a first fixture being a tubular sharp-edged tool configured to cut the element and a capturing mechanism configured to capture the element. The tubular sharp-edged tool can be further configured to be releasably attached to the instrument body.

In any of the embodiments disclosed herein, the capturing mechanism can comprise at least two tines and can be configured to be releasably attached to the biasing mechanism.

In any of the embodiments disclosed herein, the biasing mechanism can be further configured to retract the cutting and capturing assembly upon reception of a retraction signal.

In any of the embodiments disclosed herein, the cutting and capturing assembly can be further configured to retain the element. An element cut by the cutting and capturing assembly can be displaced from a cut location and at least a portion of the element may be retained within the instrument body upon reception of a retraction signal by the biasing mechanism.

In another exemplary embodiment of the present invention, a device can comprise an instrument body, a cutting and capturing assembly, and a biasing mechanism. The cutting and capturing assembly can be configured to cut an element, capture the element, and displace the element. At least a portion of the cutting and capturing assembly can be within the instrument body. The biasing mechanism can be configured to at least extend the cutting and capturing assembly upon reception of an activation signal.

In any of the embodiments disclosed herein, the instrument body can comprise at least one end configured to attach and release fixtures.

In any of the embodiments disclosed herein, the cutting and capturing assembly can comprise at least two fixtures. A first fixture can be a tubular sharp-edged tool. A second feature can be a capturing mechanism that may comprise at least two tines. The tubular sharp-edged tool can be configured to be releasably attached to the instrument body.

In any of the embodiments disclosed herein, the capturing mechanism can be further configured to be releasably attached to the biasing mechanism.

In any of the embodiments disclosed herein, the biasing mechanism can be further configured to be releasably attached to the instrument body. The biasing mechanism can further comprise an apparatus configured to be releasably attached to the biasing mechanism and receive the activation signal.

In any of the embodiments disclosed herein, the cutting and capturing assembly can be further configured to bias between at least three states upon reception of the activation signal.

In any of the embodiments disclosed herein, the cutting and capturing assembly can comprise at least two fixtures, wherein a first fixture may be a tubular sharp-edged tool and the second feature may be a capturing mechanism. The cutting and capturing assembly can be further configured to operate in a first retracted state that may enable at least the first fixture to be available.

In any of the embodiments disclosed herein, the cutting and capturing assembly can comprise at least two fixtures, wherein a first fixture may be a tubular sharp-edged tool and the second feature may be a capturing mechanism. The cutting and capturing assembly can be further configured to operate in a second state that may enable the first fixture and second fixture to be available.

In any of the embodiments disclosed herein, the cutting and capturing assembly can comprise at least two fixtures, wherein a first fixture may be a tubular sharp-edged tool and the second feature may be a capturing mechanism. The cutting and capturing assembly can be further configured to operate in a third state that may enable the first fixture and at least a portion second fixture to be available.

In another exemplary embodiment of the present invention, an interchangeable biopsy punch device may comprise a longitudinal housing that may comprise a first end. The first end may comprise an internal mechanical fixture and a second end that can be compatible with interchangeable components. The interchangeable biopsy punch device may comprise a linear actuator, which may comprise a ledge adapted to the first end of the housing, a detachable tubular cutlass, which may comprise a first end that can adapt to the second end of the housing and a second end that may comprise a blade. The interchangeable biopsy punch device may comprise an extendable prong within the housing, which can include a first end adapting into the linear actuator and a second end that may comprise a plurality of members. The interchangeable biopsy punch device may comprise a detachable cap that can adapt to the linear actuator and a coiled spring that may be proximate to the first end of the extendable prong. The coiled spring may be entrapped between the linear actuator ledge and the internal mechanical fixture proximate the first end of the longitudinal housing.

In any of the embodiments disclosed herein, the first end of the detachable tubular cutlass can be adapted to the second end of the longitudinal housing through compatible threading. The second end of the detachable tubular cutlass can be curved.

In any of the embodiments disclosed herein, the first end of the extendable prong can be adapted to the linear actuator through compatible threading. The second end of the detachable tubular cutlass can be configured to create an incised tissue sample.

In any of the embodiments disclosed herein, the linear actuator can be configured to release the extendable prong upon reception of an activation signal by the linear actuator.

In any of the embodiments disclosed herein, the coiled spring can be configured to receive the activation signal from the linear actuator ledge at the first end of the extendable prong.

In any of the embodiments disclosed herein, the second end of the extendable prong can be configured to move along the longitudinal axis of the longitudinal housing proximally to an incised tissue sample.

In any of the embodiments disclosed herein, the linear actuator can be configured to retract the extendable prong upon reception of a retraction signal by the linear actuator.

In any of the embodiments disclosed herein, the second end of the extendable prong can be further configured to remove and retain an incised tissue sample within the longitudinal housing upon reception of a retraction signal to the linear actuator by the detachable cap.

In any of the embodiments disclosed herein, the second end of the extendable prong can be further configured to eject an incised tissue sample from the longitudinal housing upon reception of an activation signal from the linear actuator.

These and other objects, features, and advantages of the present invention will become more apparent upon reading the following specification in conjunction with the accompanying drawings. These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIGS. 1A-1C show sectional and non-sectional views of the device in a retracted state according to an exemplary embodiment of the present invention.

FIGS. 2A-2C show sectional and non-sectional views of the device in an actuated state according to an exemplary embodiment of the present invention.

FIGS. 3A-3C show sectional and non-sectional views of the device in a semi actuated state according to an exemplary embodiment of the present invention.

FIGS. 4A-4E show sectional and non-sectional views of the push button of the device according to an exemplary embodiment of the present invention.

FIGS. 5A-5F show sectional and non-sectional views of a top cap for the device according to an exemplary embodiment of the present invention.

FIGS. 6A-6B show views of a claw assembly for the device according to an exemplary embodiment of the present invention.

FIGS. 7A-7D show sectional and non-sectional views of an instrument body of the device according to an exemplary embodiment of the present invention.

FIGS. 8A-8F show sectional and non-sectional views of a cutting tip of the device according to an exemplary embodiment of the present invention.

FIGS. 9A-9D show multiple sectional and non-sectional exploded views of the device according to an exemplary embodiment of the present invention.

FIGS. 10A-10B show the device during a tissue excision procedure above a target tissue sample according to an exemplary embodiment of the present invention.

FIGS. 11A-11B show the device during a tissue excision procedure being pressed against a target tissue sample according to an exemplary embodiment of the present invention.

FIGS. 12A-12B show the device during a tissue excision procedure wherein the device is removed from the tissue sample after creating an incision in a target tissue sample according to an exemplary embodiment of the present invention.

FIGS. 13A-13B show the device during a tissue excision procedure wherein the claw assembly is released toward a targeted portion of tissue within an incision of a target tissue sample according to an exemplary embodiment of the present invention.

FIGS. 14A-14B show the device during a tissue excision procedure wherein the claw assembly surrounds a targeted portion of tissue within an incision of a target tissue sample according to an exemplary embodiment of the present invention.

FIGS. 15A-15B show the device during a tissue excision procedure wherein the claw assembly retains a targeted portion of tissue from an incision of a target tissue sample and the device is removed from the target tissue sample according to an exemplary embodiment of the present invention.

FIGS. 16A-16B show the device during a tissue excision procedure wherein the claw assembly releases a targeted portion of tissue obtained from an incision of a target tissue in a separate location away from the target tissue sample according to an exemplary embodiment of the present invention.

FIGS. 17A-17B show the device during a tissue excision procedure wherein a targeted portion of tissue from an incision of a target tissue sample is retained within the device without the aid of the claw assembly according to an exemplary embodiment of the present invention.

FIGS. 18A-18B show the device during a tissue excision procedure wherein a targeted portion of tissue from an incision of a target tissue sample is expelled from the device by the claw assembly in a separate location away from the target tissue sample according to an exemplary embodiment of the present invention.

### DETAILED DESCRIPTION

The following detailed description should be read with reference to the drawings, in which like elements in different drawings are identically numbered. This description enables one skilled in the art to make and use the invention, and describes several embodiments, adaptations, variations, alternatives, and uses of the invention, including what is presently believed to be the best mode of carrying out the invention. Any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those skilled in the pertinent art in view of the teachings herein. Such modifications and variations are intended to be included within the scope of the claims.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named.

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose.

By “comprising” or “containing” or “including” is meant that at least the named compound, element, particle, or method step is present in the composition or article or method, but does not exclude the presence of other compounds, materials, particles, method steps, even if other such compounds, material, particles, method steps have the same function as what is named.

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described as making up the various elements of the invention are intended to be illustrative and not restrictive. Many suitable materials that would perform the same or a similar function as the materials described herein are intended to be embraced within the scope of the invention. Such other materials not described herein can include, but are not limited to, for example, materials that are developed after the time of the development of the invention.

For illustrative purposes, examples of the present disclosure may be directed toward surgical systems and methodologies to describe various embodiments of the device (100). However, it should be appreciated that the utility of the device (100) within the present disclosure is not solely limited to these applications. The device (100) of the present disclosure can assist in incising, extracting, capturing, displacing, and retaining tissue samples. In an exemplary embodiment of the device (100), the device (100) may comprise a push button (200), a top cap (300), an instrument body (400), a cutting and capturing assembly (500,600), which can comprise a cutting tip (500) and a claw assembly (600), and a spring (700).

The push button (200) of the device (100) may comprise a button top chamfer (201), a button lip (202), an inner threaded section (203) and a shaft (204). As one skilled in the art will appreciate, the push button (200) can be understood as a type of biasing mechanism. Biasing mechanisms can apply an input into a device or system to affect the output behavior of said device or system. Examples of biasing mechanisms can include but are not limited to switches, buttons, cams, sliders, and the like known in the art. It should be appreciated that a degree of the button top chamfer (201), a size and diameter of the button lip (202), a depth of the threading in the inner threaded section (203), dimensions of the inner threaded section (203) and dimensions of the shaft (204) of the push button (200) can be user configurable, based on a desired design specification. In some embodiments, the push button (200) of the device (100) may be configured to transition the device (100) between one or more states via application of a downward force applied uniformly to the button top chamfer (201).

The top cap (300) of the device (100) may comprise a cap knurling (301), a cap through hole (302), and threading (303) In some embodiments, the threading (303) of the top cap (300) may be male threading and be disposed below the cap knurling (301) of the top cap (300). For example, the top cap (300) can be configured to cooperatively mate with the instrument body (400) via compatible mating of the male threading (303) of the top cap (300) with female threading of the instrument body (400). It should be appreciated that a depth of the threading (303), a pitch of the cap knurling (301), and a diameter of the cap through hole (302) can be user configurable, based on a desired design specification.

The instrument body (400) of the device (100) may comprise knurled sections (401), a non-knurled section (402), a through hole (403), a top threaded section (404), a bottom threaded section (405), and an internal ledge (406). As one skilled in the art will appreciate, knurling is a type of manufacturing process used to create a pattern on a surface by displacing or deforming the surface. The knurled sections (401) of the instrument body (400) can be advantageous for a practitioner as they provide ergonomic support for gripping the device (100) while in use. It should be appreciated that pitches of the knurled sections (401) can be user configurable, based on a desired design specification.

The non-knurled section (402) of the instrument body (400) may be a section of the instrument body (400) that does not have any knurled texture pattern. In some embodiments, the non-knurled section (402) may be disposed between the knurled sections (401). It should be appreciated, that placement of the knurled sections (401) in relation to the non-knurled section (402) may be user configurable, based on a desired design specification.

The top threaded section (404) and the bottom threaded section (405) of the instrument body (400) can be disposed on a first end and a second of the instrument body (400), respectively. In some embodiments, both the top threaded section (404) and the bottom threaded section (405) may be configured with uniform female threading. The top threaded section (404) and the bottom threaded section (405) may also be configured to cooperatively mate with fixtures of the device (100) that are configured with male threading. It should be appreciated that a depth of the threading for the top threaded section (404), a diameter of the top threaded section (404), a depth of the threading for the bottom threaded section (405), and a diameter of the bottom threaded section (405) can be user configurable, based on a desired design specification.

The through hole (403) of the instrument body (400) can extend throughout the length of the instrument body (400). In some embodiments, the through hole (403) may extend from the top threaded section (404) to the bottom threaded section (405) with a diameter equal to a diameter of the top threaded section (404) and a diameter of the bottom threaded section (405) The diameter of the through hole (403) can be user configurable, based on a desired design specification.

The internal ledge (406) of the instrument body (400) can be disposed within the instrument body (400). In some embodiments, the internal ledge (406) may be disposed proximally to the top threaded section (404) of the instrument body and distally from the bottom threaded section (405) of the instrument body (400). The internal ledge (406) may also circumferentially protrude within the instrument body (400), having a diameter that can be smaller than a diameter of the top threaded section (404) or the bottom threaded section (405). It should be appreciated that a length that the internal ledge (406) protrudes within the instrument body (400), a position of the internal ledge (406) within the instrument body (400), and a shape of the internal ledge (406) can be user configurable, based on a desired design specification.

The cutting tip (500) of the cutting and capturing assembly (500, 600) may comprise a sharpened outer edge (501), a knurled section (502), a threaded section (503), and a through hole (504). In some embodiments the sharpened outer edge (501) of the cutting tip (500) may be a tubular blade, cylindrical sharpened outer edge, and the like that can be configured to make an incision in a tissue sample. In some embodiments, the sharpened outer edge (501) of the cutting tip (500) may be a blunt edge, which can be configured to create incisions within a tissue sample. The sharpened outer edge (501) may be disposed beneath the knurled section (502), protruding a desired length. It should be appreciated that dimensions of the sharpened outer edge (501) of the cutting tip (500) can be user configurable, based on a desired design specification. Varying the size, shape, or dimensions of the sharpened outer edge (501) of the cutting tip (500) can be advantageous for a practitioner as it can allow the device (100) to incise different tissue sample sizes, thicknesses, and textures.

The knurled section (502) of the cutting tip (500) may be disposed between the threaded portion (503) and the sharpened outer edge (501). The knurled section (502) of the cutting tip (500) can be advantageous for a practitioner as it can provide ergonomic support for gripping the device (100), specifically while performing an incision in a tissue sample with the sharpened outer edge (501). It should be appreciated that a pitch of the knurled section (502) of the cutting tip (500) can be user configurable, based on a desired design specification.

The threaded section (503) of the cutting tip (500) may be disposed above the sharpened edge (501) and the knurled section (502). In some embodiments, the threaded portion (503) of the cutting tip (500) may be configured with male threading, wherein the male threading of the threaded section (503) of the cutting tip (500) can compatibly mate with sections of the device (100) configured with female threading. It should be appreciated that a depth of the threading in the threaded portion (503) can be user configurable, based on a desired design specification.

The through hole (504) of the cutting tip (500) can extend throughout the length of the cutting tip (500). In some embodiments, the through hole (504) may extend from the threaded section (503) to the sharpened outer edge (501) with a diameter equal to a diameter of the threaded section (503) and a diameter of the sharpened outer edge (501). It should be appreciated that the diameter of the through hole (504) of the cutting tip (500) can be user configurable, based on a desired design specification.

The claw assembly (600) of the cutting and capturing assembly (500, 600) may comprise claw tines (601), a claw crimp point (602), a threaded section (603), and a claw tubular body (604). The claw tines (601) can be configured to capture an excised tissue sample from the site. In some embodiments, the claw tines (601) may operate in a closed position, wherein the claw tines (601) are not extended, and an open position, wherein the claw tines (601) are fully extended. In some embodiments, the claw tines (601) may be tensioned and may expand in an open configuration once tension is relieved from the claw tines (601). It should be appreciated that the number of claw tines (601), shape of the claw tines (601), and the material composition of the claw tines (601) are user configurable, based on a desired design specification. In some embodiments, the claw assembly (600) may comprise two or more claw tines (601), which can be configured to capture and retain a tissue sample when performing a tissue excision procedure.

The claw crimp point (602) of the claw assembly (600) may be disposed between a first end of the claw tubular body (604) and a second end of the claw tubular body (604). In some embodiments, the claw crimp point (602) may be disposed proximal to the claw tines (601) of the claw assembly (600). As one skilled in the art will appreciate, crimping is a method of j oining one or more pieces of material together through deforming one or more pieces of material. In some embodiments, the claw crimp point (602) can be configured to be a joint mating two sections of the claw tubular body (604) of the claw assembly (600).

The threaded section (603) of the claw assembly (600) may be disposed at an end of the claw tubular body (604) opposite the claw tines (601). In some embodiments, the threaded section (603) of the claw assembly (600) may be configured with male threading. For example, male threading of the threaded section (603) of the claw assembly (600) can compatibly mate with areas of the device (100) configured with female threading. It should be appreciated that a depth of the threading in the threaded portion (603) can be user configurable, based on a desired design specification.

The claw tubular body (604) of the claw assembly may be configured with the threaded section (603) on a first end of the claw tubular body (604) and claw tines (601) on a second end of the claw tubular body (604). In some embodiments, the claw tubular body (604) may have a uniform tubular shape. It should be appreciated that a length of the claw tubular body (604) of the claw assembly (600) and a geometry of the claw tubular body (604) can be user configurable, based on a desired design specification.

The spring (700) of the device (100) may be disposed within the instrument body (400). As one skilled in the art will appreciate, mechanical springs are elastic devices that can store potential mechanical energy, when compressed, and release said potential mechanical energy, when decompressed. In some embodiments, the spring (700) may be disposed atop the internal ledge (406) of the instrument body (400), wherein the internal ledge (406) may be configured to operate as a seat for the spring (700).

In an exemplary embodiment, the components of the device (100) may be assembled as follows: (1) The cutting tip (500) can be screwed onto the bottom threaded section (405) of the instrument body (400); (2) The spring (700) can be inserted into the instrument body (400) and can be placed on the internal ledge (406) within the instrument body (400); (3) The claw tubular body (604) of the claw assembly (600) can be pushed into the instrument body (400) through the through hole (504) of the cutting tip (500) and the spring (700); (4) The push button (200) can threaded into the threaded section (603) of the claw assembly (600) via the inner threaded section (203) of the push button (200); (5) The top cap (300) can be screwed onto the top threaded portion (404) of the instrument body (400) with the button top chamfer (202) of the push button (200) passing through the through hole (302) of the top cap (300). It should be appreciated that the push button (200), top cap (300), instrument body (400) and cutting tip (500) can all be made of surgical grade stainless steel, or another suitable material commonly known within the art.

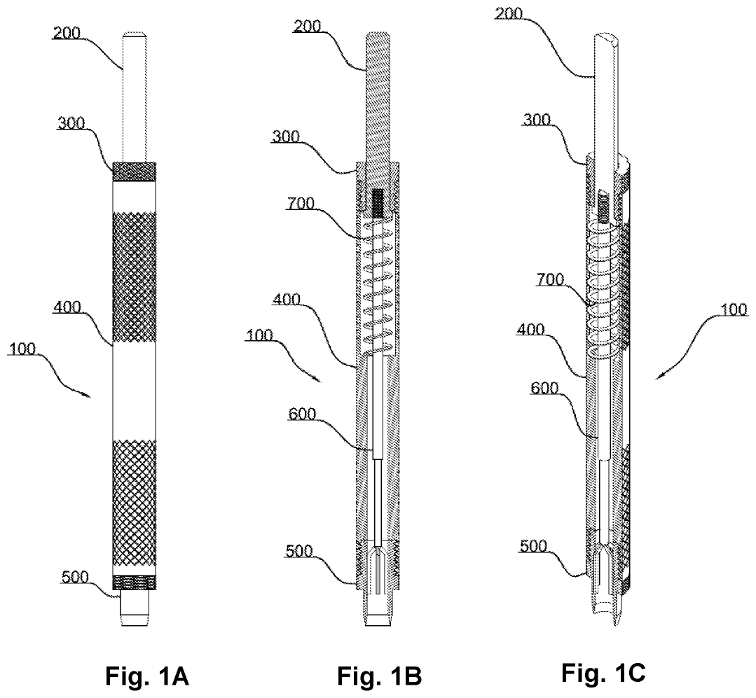

As shown in FIGS. 1-3, the device (100) may be configured to operate in one or more states upon depressing the push button (200). FIGS. 1A-1C illustrate sectional and non-sectional views of a retracted state for the device (100). The device (100) can remain in the retracted state due to the push button (200) not being depressed. The push button, when not depressed, can prevent the extension of the claw assembly (600) of the cutting and capturing assembly (500, 600). In some embodiments, at least a portion of the cutting and capturing assembly (500,600) can remain within the instrument body (400) of the device (100). For example, as shown in FIGS. 1A-1C, the claw assembly (600) of the cutting and capturing assembly (500, 600) may remain within the instrument body (400) while in the retracted state. The device (100) can transition to the retracted state by removing any downward force exerted on the push button (200). Once the downward force is removed from the push button (200), the spring (700) may expand and push up against the button lip (202) of the push button (200) and the internal ledge (406) of the instrument body (400) simultaneously. The expansion of the spring (700) against the button lip (202) of the push button (200) can retract at least a portion of the cutting and capturing assembly (500, 600) within the instrument body (400) in an upward motion through the through hole (504) of the cutting tip (500).

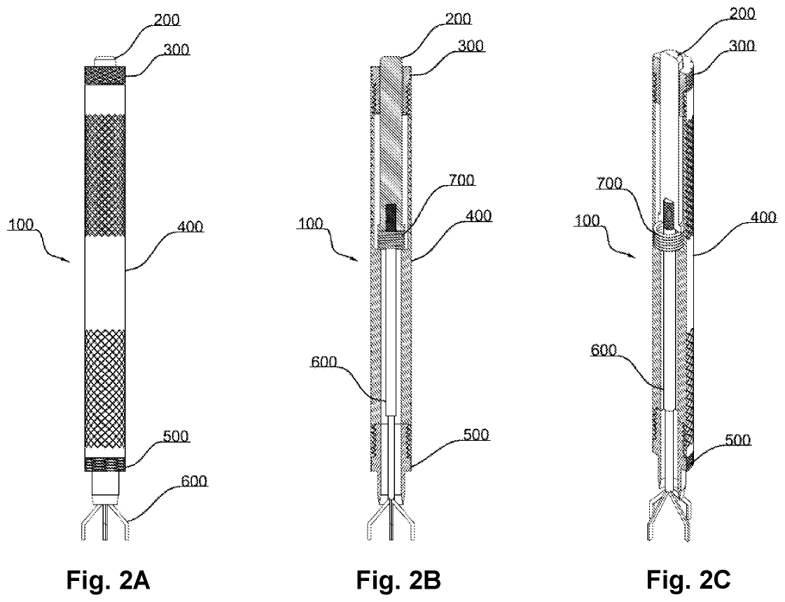

FIGS. 2A-2C shows sectional and non-sectional views of the device (100) in an actuated state for the device (100). In some embodiments, the device (100) while operating in the actuated state, may have all fixtures of the cutting and capturing assembly (500, 600) available for use. For example, the device (100) while in the actuated state can allow the practitioner to utilize both the cutting tip (500) and the claw mechanism (600) simultaneously. The device (100) may achieve the actuated state through applying a downward force to the button top chamfer (201) of the push button (200). The push button (200), after receiving the downward force, may compress the spring (700) between the button lip (202) and the internal ledge (406), thus extending the claw assembly (600) of the cutting and capturing assembly (500, 600) through the through hole (504) of the cutting tip (500) and thereby the instrument body (400). While extended, the claw assembly (600) may extend the claw tines (601), which upon moving through the through hole (503) cutting tip (500) may expand in an open fashion once fully through the through hole (503) of the cutting tip (500) as shown in FIGS. 2A-2C.

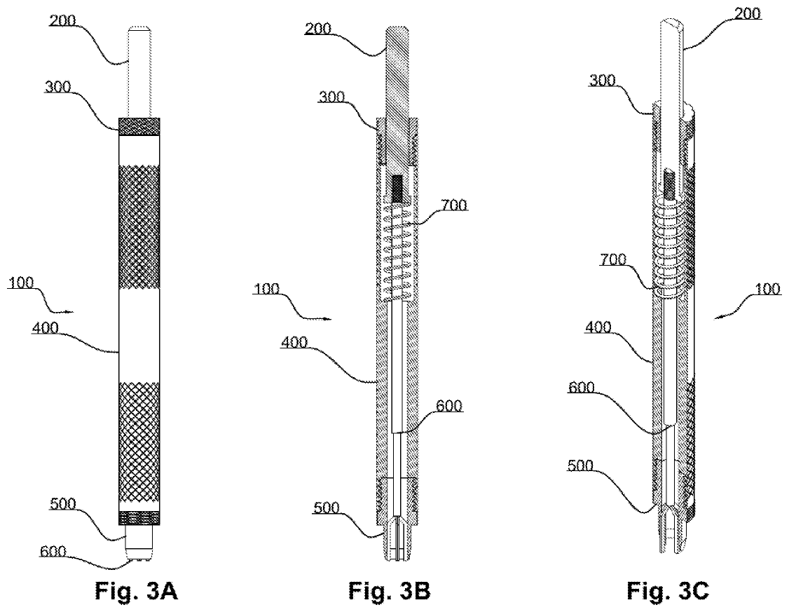

FIGS. 3A-3C show sectional and non-sectional views of the device (100) in a semi actuated state. In contrast to the illustrations shown in FIGS. 1-2, FIGS. 3A-3C shows a semi actuated state for the device (100), wherein the cutting tip (500) and a portion of the claw assembly (600) of the cutting and capturing assembly (500, 600) can be available for use. In some embodiments, while in the semi actuated state, the push button (200) may not be fully compressed, as shown in FIGS. 3A-3C. Furthermore, while the device (100) is in the semi actuated state, the claw tines (601) of the claw assembly (600) can be in a more closed fashion, such as what is shown in FIGS. 3A-3C. It should be appreciated that the arrangement of the claw tines (601) of the claw assembly (600), while in the semi actuated state, can allow the practitioner to both retain an excised tissue sample within the claw assembly (600) and displace said excised tissue sample to a separate location.

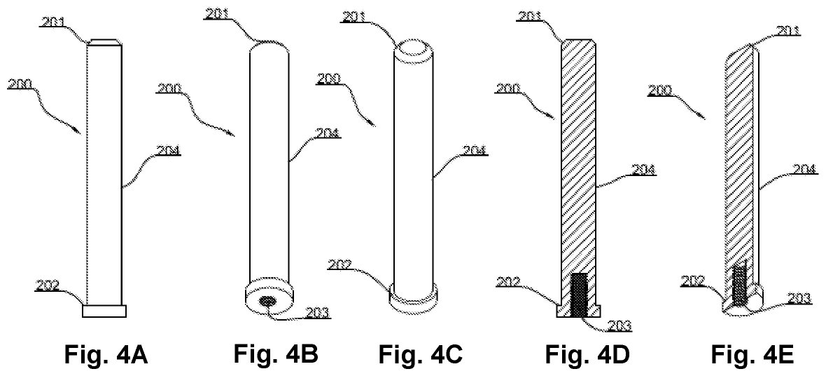

Referring now to FIGS. 4A-4E, multiple sectional and non-sectional views of the push button (200) of the device (100) and elements of the push button (200) are shown.

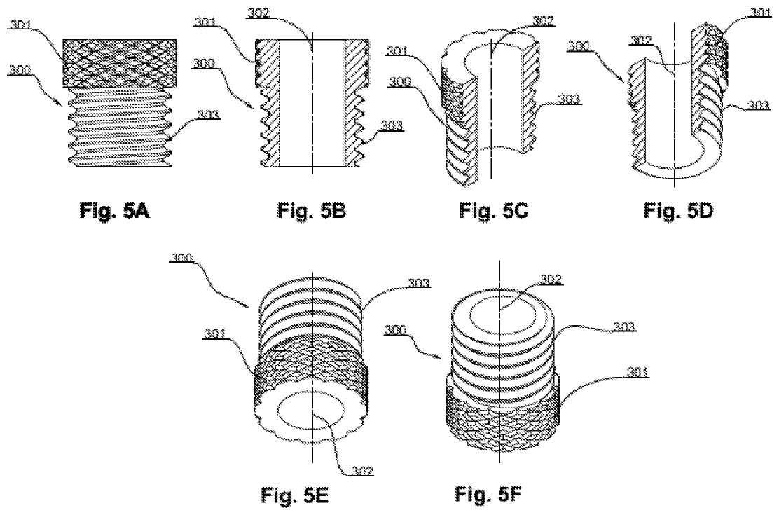

Referring now to FIGS. 5A-5F, multiple sectional and non-sectional views of the top cap (300) for the device (100) and elements of the top cap (300) are shown.

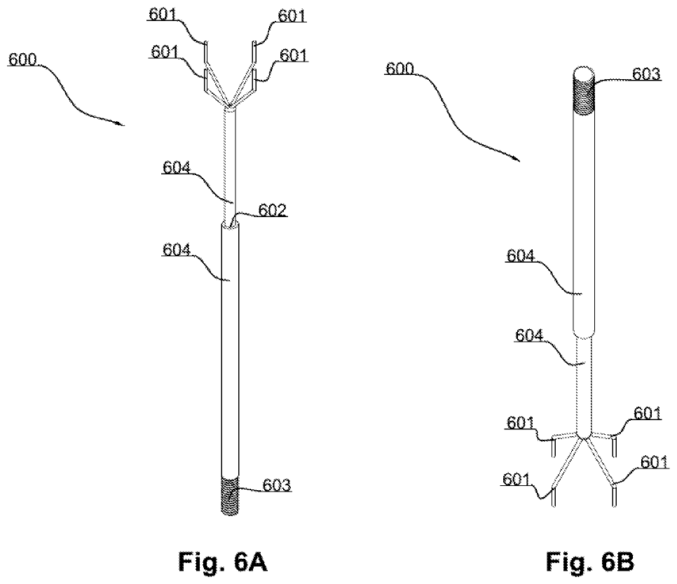

Referring now to FIGS. 6A-6B, two views of the claw assembly (600) of the cutting and capturing assembly (500,600) and elements of the claw assembly (600) are shown. In some embodiments, the claw tubular body (604), as shown in FIGS. 6A-6B, may have a first section with a larger diameter that can be joined to a second section of the claw tubular body (604) via the claw crimp point (602).

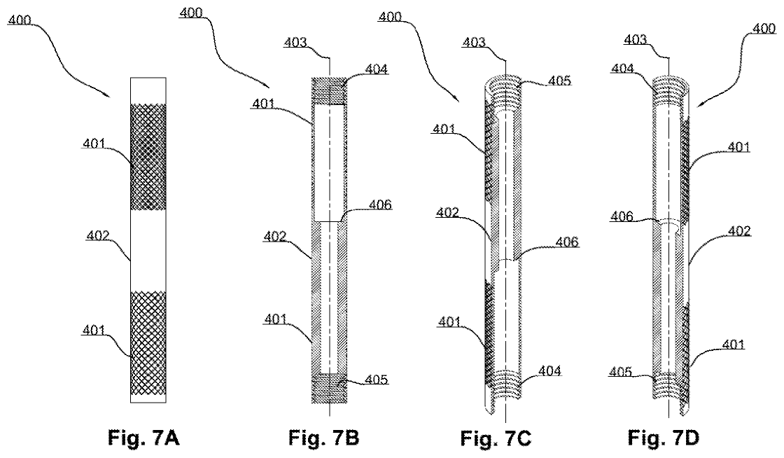

Referring now to FIGS. 7A-7D, multiple sectional and non-sectional views of the instrument body (400) of the device (100) and elements of the instrument body (400) are shown.

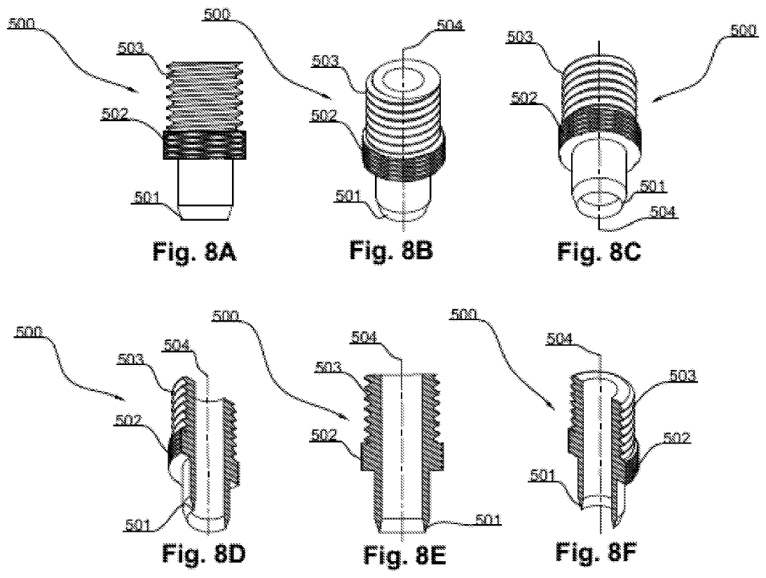

Referring now to FIGS. 8A-8F, multiple sectional and non-sectional views of the cutting tip (500) of the device (100) and elements of the cutting tip are shown.

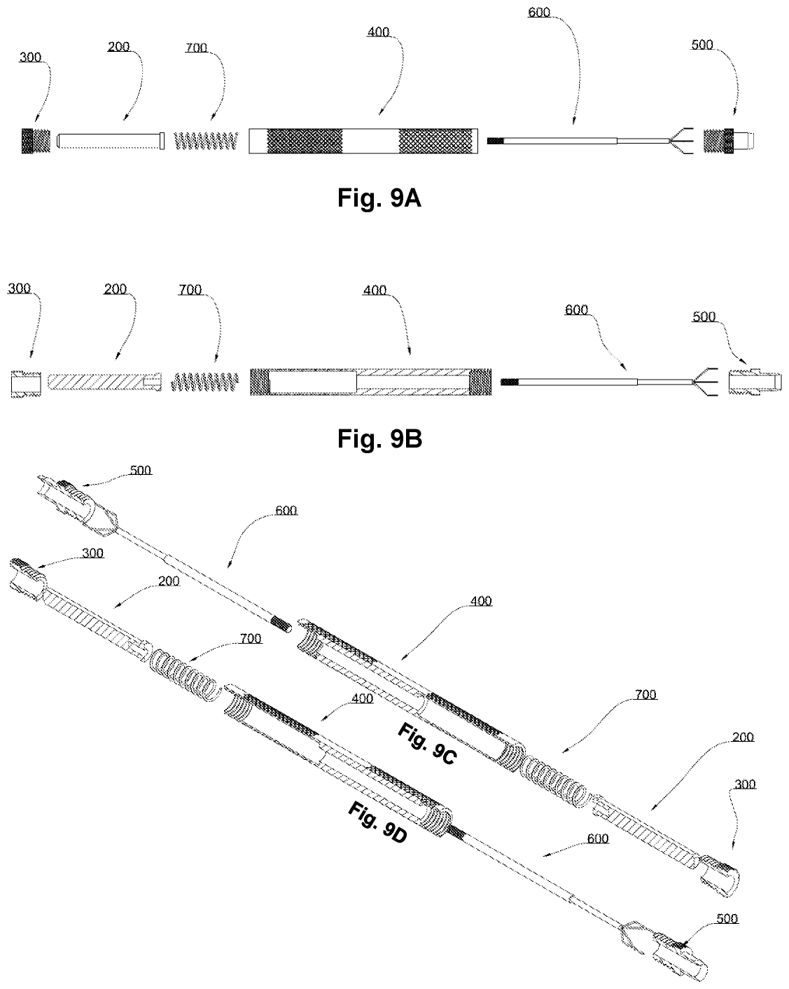

Referring now to FIGS. 9A-9D, multiple sectional and non-sectional exploded views of the device (100) and the elements of the device (100) are shown.

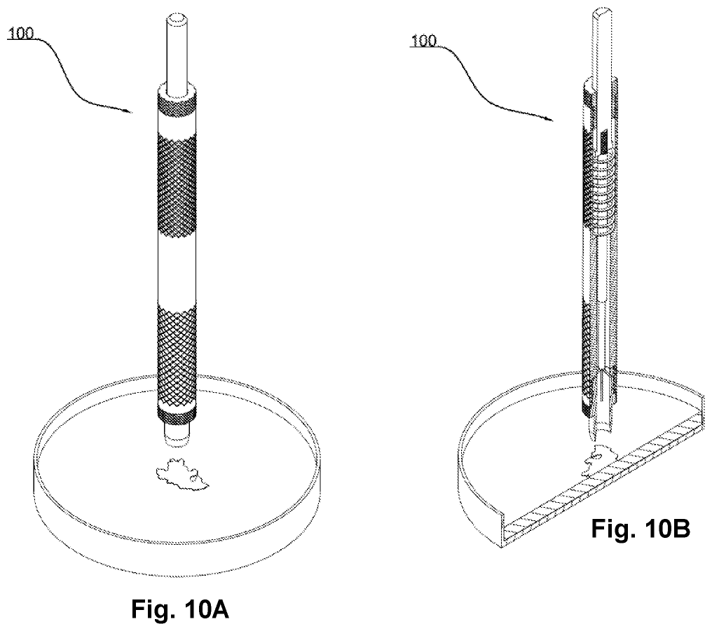

FIGS. 10A-10B show the device during tissue excision above a target tissue sample. In some embodiments, prior to contacting the target tissue sample, the device (100) may begin in the retracted state. In some embodiments, while the device (100) is in the retracted state, the cutting and capturing assembly (500, 600) may only have the cutting tip (500) available for use, as shown in FIGS. 10A-10B. During a tissue excision procedure, for example, the device (100) may be firmly grasped by the practitioner at either of the one or more knurled sections (401) of the instrument body (400).

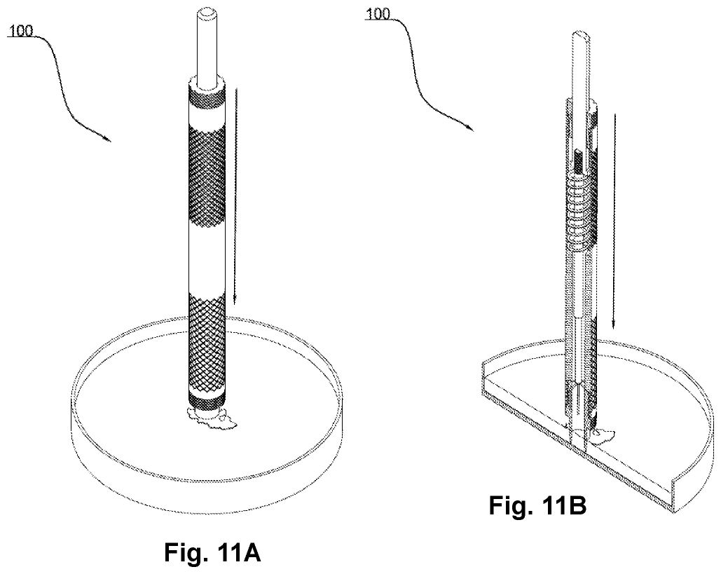

FIGS. 11A-11B show the device (100) during a tissue excision procedure being pressed against a target tissue sample. In some embodiments, the device (100) may be pressed against the target tissue sample, wherein the practitioner may roll the device (100) between their thumb and index fingers. The practitioner may perform this action with the device (100) until an incision is made in the tissue sample, which can have a maximum depth of the vertical length of the sharpened outer edge (501) of the cutting tip (500). As should be appreciated, the cutting tip (500) of the device (100) may reach the knurled section (502) of the cutting tip (500) when fully submerged in the target tissue sample.

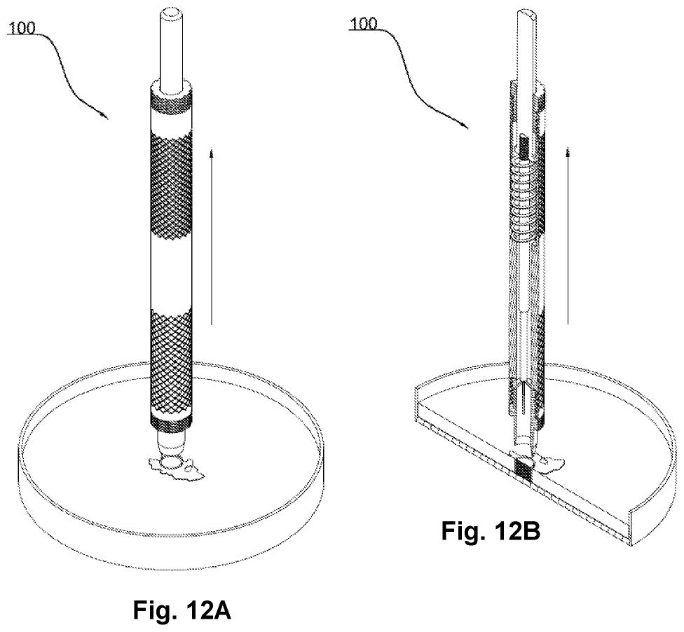

FIGS. 12A-12B show the device (100) during a tissue excision procedure wherein the device (100) is removed from the tissue sample after creating an incision in a target tissue sample. Once the incision is made, the device (100) can be fully pulled out of the incision. The practitioner may adjust their grip such that their thumb can be on one side of the one or more knurled sections (401) of the instrument body (400) and their middle finger can be on the other side. Based on this grip position, the practitioner may position their index finger to prepare to operate the push button (200), which can engage the cutting and capturing assembly (500, 600).

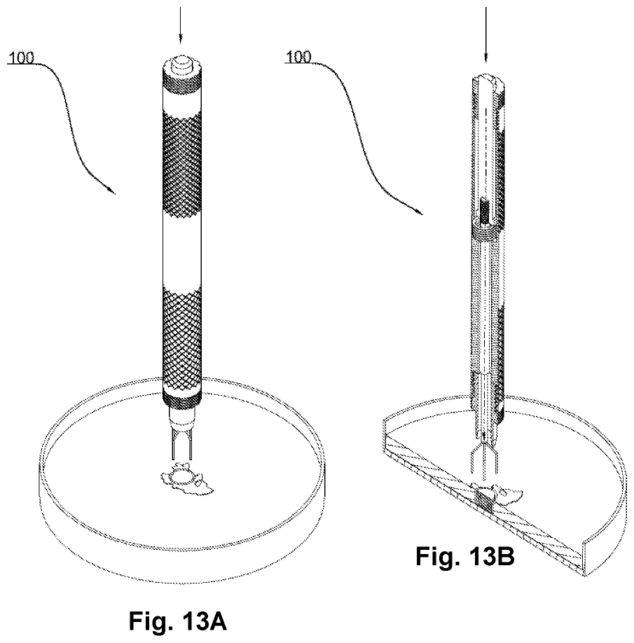

FIGS. 13A-13B show the device (100) during a tissue excision procedure wherein the claw assembly (600) is released toward a targeted portion of tissue within an incision of a target tissue sample Once the device (100) is oriented in the practitioner's hand, the practitioner can now depress the push button (200) and thereby compress the spring (700) to engage the cutting and capturing assembly (500, 600). Once the push button (200) is fully depressed, as shown in FIGS. 13A-13B, the claw assembly (600) may extend through the instrument body (400) and the through hole (502) of the cutting tip (500). It should be appreciated that once the claw assembly (600) extends through the through hole (502) of the cutting tool (500), the claw tines (601) may open, and the device (100) can enter the actuated state.

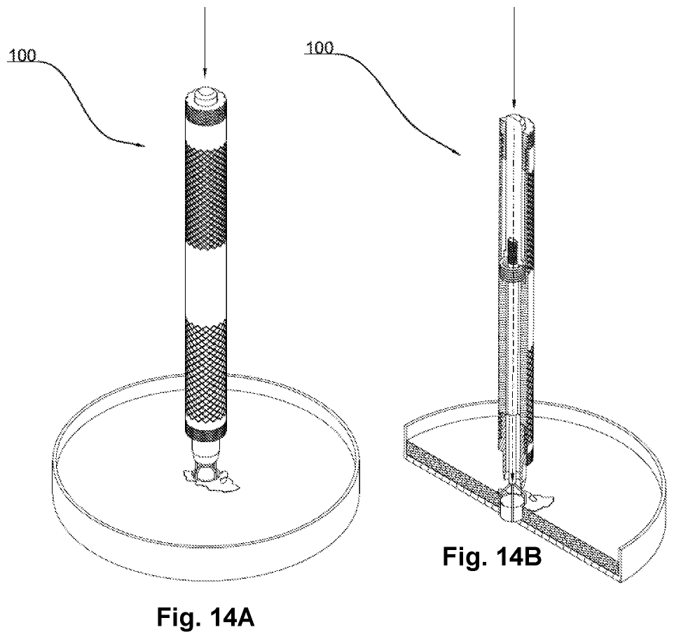

FIGS. 14A-14B show the device (100) during a tissue excision procedure wherein the claw assembly (600) surrounds a targeted portion of tissue within an incision of a target tissue sample. In some embodiments, the practitioner may depress the push button (200) until the claw tines (601) are submerged in the incision. It should be appreciated that the claw tines (601) of the claw assembly (600) may surround the targeted portion of tissue to be extracted, which can be beneficial in ensuring a secure grip of the targeted portion of tissue.

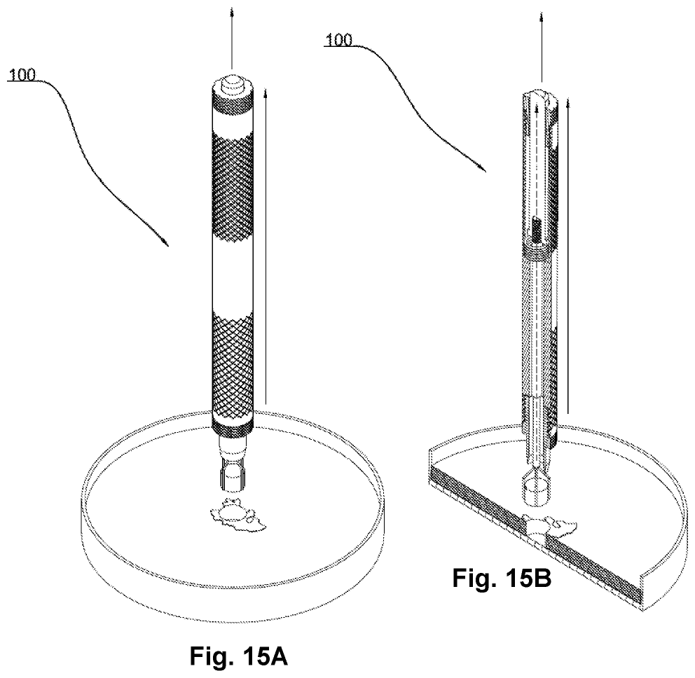

FIGS. 15A-15B show the device (100) during a tissue excision procedure wherein the claw assembly (600) retains a targeted portion of tissue from an incision of a target tissue sample and the device (100) is removed from the target tissue sample. As shown in FIGS. 15A-15B, the device (100) can operate in the semi actuated state once the targeted portion of tissue is secured in the claw tines (601) of the claw assembly (600). To transition to the semi-actuated state, the downward force exerted on the push button (200) is relieved, which can allow the spring (700) to expand against the button lip (200) and therein retract the claw assembly (600) It should be appreciated that while in the semi actuated state, the device (100) may have at least a portion of the claw assembly (600) and the cutting tool (500) of the cutting and capturing assembly (500,600) available.

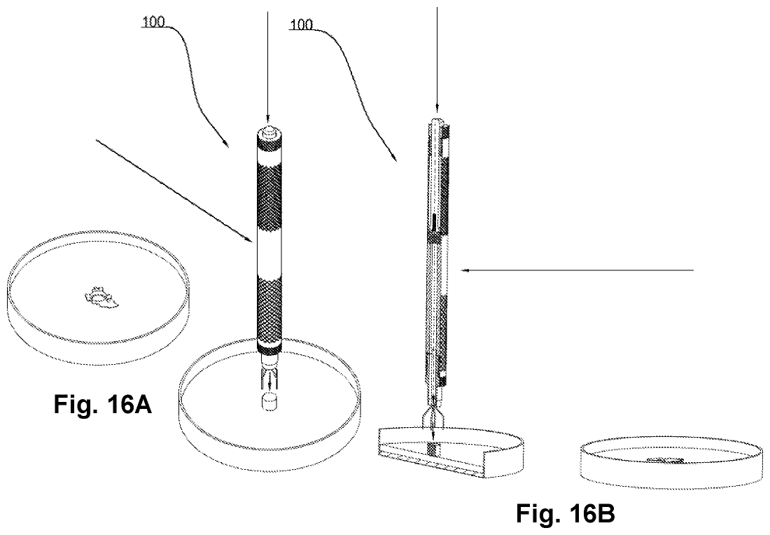

FIGS. 16A-16B shows the device (100) during a tissue excision procedure wherein the claw assembly (600) releases a targeted portion of tissue obtained from an incision of a target tissue sample in a separate location. In some scenarios, the practitioner may desire to move the targeted portion of tissue from an incision of a target tissue sample. As shown in FIGS. 16A-16B, the practitioner may place the targeted portion of tissue in a separate location, such as a Petri dish, microscope slide, or the like, to perform further analysis on the excised tissue sample.

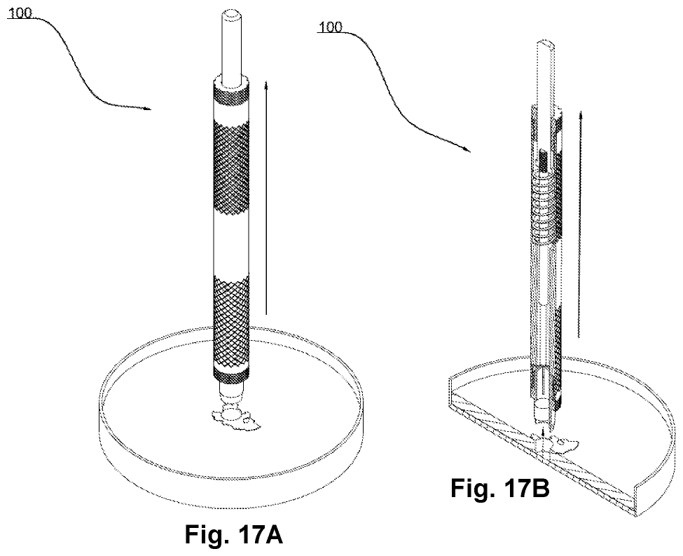

FIGS. 17A-17B show the device (100) during a tissue excision procedure wherein a targeted portion of tissue from an incision of a target tissue sample is retained within the device (100) without the aid of the claw assembly (600). In some embodiments, the device (100) may incise a targeted tissue sample in the retracted state. While in the retracted state, the device (100) may only have the cutting tip (500) of the cutting and capturing assembly (500,600) available as the push button (200) is not depressed. Given that the push button (200) is not depressed, the claw assembly (600) of the cutting and capturing assembly (500, 600) can remain disengaged. As mentioned previously, the practitioner may roll the device (100) between their thumb and index finger until an incision is made in the tissue sample, which can have a maximum depth of a vertical length of the sharpened outer edge (501) of the cutting tip (500). It should be appreciated that the surface area of the targeted tissue sample may wedge itself within the through hole (504) of the cutting tip (500), which may allow the device (100) to retain the targeted tissue sample without the aid of the claw assembly (600).

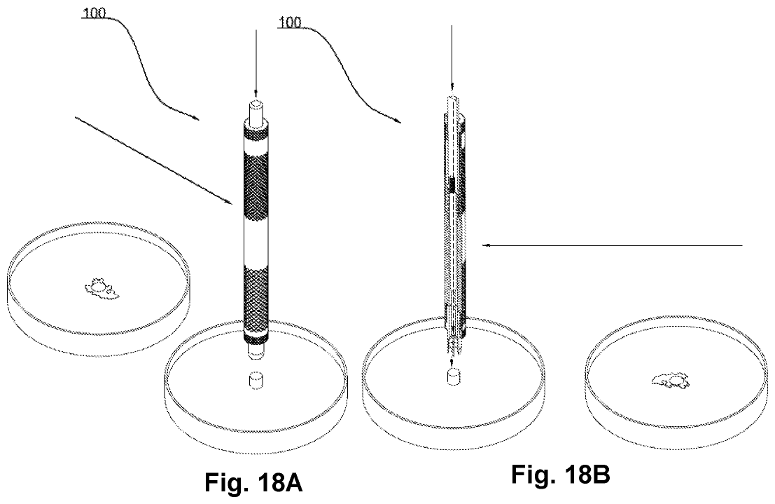

FIGS. 18A-18B show the device during a tissue excision procedure wherein a targeted portion of tissue from an incision of a target tissue sample is expelled from the device (100) by the claw assembly (600) in a separate location away from the target tissue sample. In contrast to the claw assembly (600) being used to capture and retain the targeted portion of tissue, the claw assembly (600) may also be used like a plunger, expelling the targeted portion of tissue wedged in the through hole (504) of the cutting tip (500) from the device (100). In some embodiments, the claw assembly (600) can expel the retained target tissue sample once a downward force is exerted on the push button (200). Once the push button (200) is depressed, the claw assembly (600) can extend toward the through hole (504) of the cutting tip (500) and contact the retained target tissue sample with the claw tines (601), thus expelling the retained target tissue sample.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

What is claimed is:

1. A device comprising:
an instrument body;
a cutting and capturing assembly configured to cut an element, capture the element, and displace the element wherein at least part of the cutting and capturing assembly is within the instrument body and;
a biasing mechanism configured to at least extend the cutting and capturing assembly upon reception of an activation signal.

2. The device of claim 1, wherein the instrument body comprises at least one end configured to attach and release fixtures.

3. The device of claim 1, wherein the cutting and capturing assembly is further configured to bias between at least three states upon reception of the activation signal by the biasing mechanism.

4. The device of claim 1 further comprising an apparatus configured to releasably attach to the biasing mechanism.

5. The device of claim 1, wherein the biasing mechanism is further configured to be releasably attached to the instrument body.

6. The device of claim 1, wherein the element cut by the cutting and capturing assembly is displaced from a cut location and retained within the instrument body.

7. The device of claim 1, wherein the cutting and capturing assembly comprises at least two fixtures, a first fixture being a tubular sharp-edged tool and a second fixture being a capturing mechanism comprising at least two tines.

8. The device of claim 7, wherein the capturing mechanism is configured to releasably attach to the biasing mechanism.

9. The device of claim 7, wherein the tubular sharp-edged tool is configured to releasably attach to the instrument body.

10. The device of claim 3, wherein the cutting and capturing assembly is further configured to operate in a first retracted state where at least the tubular sharp-edged tool is available.

11. The device of claim 3, wherein the cutting and capturing assembly is further configured to operate in a second actuated state of the at least three states where the tubular sharp-edged tool and capturing mechanism are available.

12. The device of claim 3, wherein the cutting and capturing assembly is further configured to operate in a third semi-actuated state of the at least three states where the tubular sharp-edged tool and at least a portion of the capturing mechanism are available.
